(19) United States
(12) Patent Application Publication
(10) Pub. No.: US 2024/0038330 A1
(43) Pub. Date: Feb. 1, 2024

(54) COMPUTER-IMPLEMENTED METHOD AND APPARATUS FOR ANALYSING GENETIC DATA

(71) Applicant: GENOMICS PLC, Oxford (GB)

(72) Inventors: Rachel MOORE, Oxford (GB); Vincent Yann Marie PLAGNOL, Oxford (GB); Fernando RIVEROS-MCKAY, Oxford (GB); Michael WEALE, Oxford (GB); Daniel WELLS, Oxford (GB); Christopher Charles Alan Spencer, Oxford (GB)

(73) Assignee: GENOMICS PLC, Oxford (GB)

(21) Appl. No.: 18/255,249

(22) PCT Filed: Nov. 26, 2021
(86) PCT No.: PCT/GB2021/053068

§ 371 Date: May 31, 2023

(30) Foreign Application Priority Data
Dec. 1, 2020 (GB) 2018904.9

(51) Int. Cl. G16B 20/20; G16B 40/20

(52) U.S. Cl. CPC G16B 20/20; G16B 40/20

(57) ABSTRACT

Disclosed is a method of analysing genetic data about an organism comprising receiving a plurality of input units. Each input unit comprises information about the association between genetic variants in a region of the genome and a target phenotype. One or more iterations are carried out comprising, for each variant, determining whether the variant is causal for the target phenotype. If the variant is causal, a sampled effect size is determined for each input unit based on the input units and correlations between the plurality of genetic variants in the region. The sampled effect size is non-zero for all of the input units. For each variant, a prediction effect size is determined for each input unit based on an average across the iterations of the sampled effect sizes for the input unit or of posterior effect sizes for the input unit calculated using the sampled effect sizes.

The invention relates to analysing genetic and phenotype data about an organism to obtain information about the organism, particularly in the context of enabling improved polygenic risk scores (PRSs) to be obtained for phenotypes of interest.

A PRS is a quantitative summary of the contribution of an organism's inherited DNA to the phenotypes that it may exhibit. A PRS may include in its computation all DNA variants relevant (either directly or indirectly) to a phenotype of interest or may use its component parts if these are more relevant to a particular aspect of an organism's biology (including cells, tissues, or other biological units, mechanisms or processes). A PRS can be used directly, or as part of a plurality of measurements or records about the organism, to infer aspects of its past, current, and future biology.

PRSs are gaining traction as a tool for disease prevention, stratification and diagnosis. In the context of improving human health and healthcare, PRSs have a range of practical uses, which include, but are not limited to: predicting the risk of developing a disease or phenotype, predicting age of onset of a phenotype, predicting disease severity, predicting disease subtype, predicting the response to treatment, selecting appropriate screening strategies for an individual, selecting appropriate medication interventions, and setting prior probabilities for other prediction algorithms.

PRS may have direct use as a source of input in the application of artificial intelligence and machine learning approaches to making predictions or classifications from other high dimensional input data (for example imaging). They may be used to help train these algorithms, for example to identify predictive measurements based on non-genetic data. As well as having utility in making predictive statements about an individual, they can also be used to identify cohorts of individuals, included but not limited to the above applications, by calculating the PRS for a large number of individuals, and then grouping individuals on the basis of the PRSs.

PRSs can also aid in the selection of individuals for clinical trials, for example to optimise trial design by recruiting individuals more likely to develop the relevant disease or phenotypes, thereby enhancing the assessment of the efficacy of a new treatment. PRSs carry information about the individuals they are calculated for, but also for their relatives (who share a fraction of their inherited DNA). Information about the impact of an individual's DNA on their phenotypes can derive from any relevant assessment of the potential impact of carrying any particular combination of DNA variants.

In what follows we focus on the analysis of the recent wealth of information that derives from genetic association studies (GAS). These studies systematically assess the potential contribution of DNA variants to the genetic basis of a phenotype.

Since the mid-2000s, GAS (typically genome-wide association studies: GWAS, or association studies targeting single variants, or variants in a region of the genome, or GWAS restricted to a particular region of the genome) have been conducted on many thousands of (largely human) phenotypes, in millions of individuals, generating billions of potential links between genotypes and phenotypes. The resulting raw data is often then simplified to produce summary statistic data. GAS summary statistic data consists of, for each genetic variant (whether imputed or observed), the inferred effect size of the genetic variant on the phenotype of the GAS and the standard error of the inferred effect size. In other cases the individual level data, consisting of a full genetic profile of the individuals in a study and information about their phenotypes, may be available directly. However, individual level data is typically less widely available due to requirements on the privacy of an individual's data.

A PRS consists of the aggregation of the effects of a large number of genetic variants, typically each having small individual effects, to build an aggregate predictor for a trait of interest. PRSs can be calculated using effect sizes of variants determined from GWAS. Variants included in such a score can either be “causal variants”, in the sense that the variants directly affect a trait (weakly, but directly), or “tag variants”, which means that they are strongly correlated with other, unknown, variants that are causal, but that the tag variant itself does not have a direct effect on the phenotype.

Strategies for PRS construction are expanding, but a well-accepted general approach to building an accurate PRS consists of deconvoluting the signal in all regions of association by investigating the combination of variants that best capture the underlying biological associations. The number of associations will vary, with many genomic regions containing a single potential association while some genomic regions will contain multiple independent associations (up to 10 has been reported, though this is rare).

A technical challenge in identifying the correct combination of variants responsible for all the associations in a region is that these variants can be correlated with each other. The larger the correlations are, the higher the number of samples will be required to break down these correlations.

Some tools to build PRSs are designed to take advantage of summary statistics data. One approach, popularised by the LDpred software (Vilhjalmsson et al 2015, https://github.com/bvilhjal/ldpred), iterates through multiple random selections of plausible variants genome-wide based on a single GWAS and, as variants are picked or removed, estimates the residual signal.

A strength of the summary statistics data based strategy is that the absence of limitation around sharing of individual level data means that much larger sample sizes can be made available to the scientific community. This is why much of the current PRS design is based on these large summary statistics datasets.

However, for all summary statistics data based methods, correlated variants are handled by referring to an external data source describing what the correlations between variants are expected to be. The pattern of correlations between genetic variants is referred to as linkage disequilibrium (LD). A limitation of relying on an external dataset to describe the pattern of LD is that different subpopulations have distinct patterns of LD. For example, individuals of European ancestry may have different patterns of LD to individuals of South-East Asian ancestry. Given that the identity of true causal variants is usually never known with absolute certainty, these differences in LD can lead to differences in the predictive accuracy of the PRS in different ancestries. In addition, the effect of particular variants on a phenotype may vary between subpopulations. For example, a given causal genetic variant may have a larger effect on a given phenotype in men than in women, or a smaller effect in older individuals than in younger individuals. Therefore, inferences made for one subpopulation, or made based on data from individuals from a mixture of subpopulations, are unlikely to be as precise for different subpopulations. For example, the datasets that support the construction of PRSs are often based on large cohorts of European ancestries. As a result, these scores often perform poorly in non-European ancestries.

Existing methods to deal with this issue are based upon creating PRS using training datasets from the appropriate subpopulation. However, the amount of data that is available for particular subpopulations can vary greatly. Therefore, these methods suffer from much lower sample sizes, which in turn limit their predictive power. Due to the reduced statistical power of smaller studies, attempting to calculate a PRS for a particular subpopulation for which there is little data available may produce less reliable results than simply using a result obtained from a different subpopulation for which there is more data available. For example, in many cases, the larger sample size of cohorts from European ancestries can overcome the bias associated with using non-matched training sets, and a PRS trained on European ancestries may in fact provide the best PRS option in non-European cohorts, even though this is in principle sub-optimal.

It is an object of the invention to improve analysis of genetic data about an organism and/or allow more robust and/or accurate PRSs to be obtained for individuals belonging to particular subpopulations.

According to an aspect of the invention, there is provided a computer-implemented method of analysing genetic data about an organism. The method comprises receiving a plurality of input units, wherein each input unit comprises information about the association between a plurality of genetic variants in a region of interest of the genome of the organism and a target phenotype of the organism, carrying out one or more iterations comprising, for each of the plurality of genetic variants: determining whether the genetic variant is causal for the target phenotype based on the plurality of input units; and if the genetic variant is determined to be causal, determining a sampled effect size of the genetic variant on the target phenotype for each of the input units based on the plurality of input units and information about correlations between the plurality of genetic variants in the region of interest, the sampled effect size of the genetic variant on the target phenotype being non-zero for all of the input units, and for each genetic variant, determining a prediction effect size of the genetic variant on the target phenotype for each of the input units based on an average across at least a subset of the iterations of the sampled effect sizes of the genetic variant for the input unit or of posterior effect sizes of the genetic variant for the input unit calculated using the sampled effect sizes.

By determining which variants are causal using data from a plurality of input units, the causal variants can be identified with greater confidence. However, determining a prediction effect size separately for each input unit nonetheless allows the method to account for the possibility of different effect sizes for different subpopulations. Thereby, the statistical power of using large datasets can be combined with the ability to generate subpopulation-specific conclusions. By obtaining more accurate prediction effect sizes, more accurate PRS can consequently be calculated.

In some embodiments, determining whether the genetic variant is causal comprises calculating the probability of the information from the plurality of input units, assuming that the genetic variant is causal and a probability of the information from the plurality of input units assuming that the genetic variant is not causal, and stochastically determining the genetic variant to be causal with a probability dependent on a ratio of the probability of the input data assuming that the genetic variant is causal and the probability of the input data assuming that the genetic variant is not casual. Using stochastic sampling allows the method to consider many different combinations of causal variants to identify an overall effect that best explains the observed data.

In some embodiments, the probability of the information from the plurality of input units assuming that the genetic variant is causal is dependent on: a proportion of the plurality of genetic variants expected to be causal; the plurality of input units; and a correlation between the effect sizes of the genetic variant on the target phenotype for each of the input units. In some embodiments, the probability of the information from the plurality of input units assuming that the genetic variant is not causal is dependent on: a proportion of the plurality of genetic variants expected to be causal; and the plurality of input units. These terms allow pre-existing information about the proportion of variants that are causal to be incorporated in the analysis, and allow the prediction effect sizes between input units to vary. In the non-causal case, the effect sizes are zero, so no correlation between effects is appropriate.

In some embodiments, the proportion of the plurality of genetic variants expected to be causal is predetermined. In some embodiments, the correlation between the effect sizes of the genetic variant on the target phenotype for each of the input units is predetermined. Using predetermined values of the parameters allows pre-existing knowledge to be incorporated in the method in a computationally efficient manner.

In some embodiments, the proportion of the plurality of genetic variants expected to be causal is updated at each iteration. In some embodiments, wherein the correlation between the effect sizes of the genetic variant on the target phenotype for each of the input units is updated at each iteration. Learning and updating the parameters at each iteration allows the method to converge on the true parameter values that may provide a more accurate result, but may be more computationally expensive.

In some embodiments, the input units are determined from respective groups of individuals, and the probability of the information from the plurality of input units assuming that the genetic variant being causal is dependent on one or more parameters quantifying an overlap in the groups of individuals between respective pairs of input units. Depending on the data used, some individuals may be present in multiple input units, which can distort the conclusions drawn. Adding parameters to account for this improves the accuracy of the resulting effect sizes.

In some embodiments, determining the sampled effect size of the genetic variant comprises calculating a probability distribution of effect sizes of the genetic variant on the target phenotype for the input units, and sampling values of the effect sizes for the input units from the probability distribution. Using a probability distribution allows the method to sample different effect sizes, while still encouraging values to be chosen in a range considered most likely to be correct.

In some embodiments, the probability distribution is a multivariate normal distribution. Using a multivariate normal distribution provides a convenient way to allow different sampled effect sizes for different input units.

In some embodiments, the sampling of values of the effect size in each iteration is dependent on the sampled effect sizes from one or more previous iterations. This type of dependence can allow sampling to efficiently explore the space of possible values. In some embodiments, the sampling of values of the effect size is performed using a Monte-Carlo Gibbs sampler. This type of sampling algorithm is particularly suited to the present application.

In some embodiments, the probability distribution is dependent on a correlation between the effect sizes of the genetic variant on the target phenotype for each of the input units. This allows the likely range of differences in effect size between input units to be controlled to improve accuracy and computational efficiency.

In some embodiments, the correlation between the effect sizes of the genetic variant on the target phenotype for each of the input units is predetermined. Using predetermined values of the parameters allows pre-existing knowledge to be incorporated in the method in a computationally efficient manner.

In some embodiments, the correlation between the effect sizes of the genetic variant on the target phenotype for each of the input units is updated at each iteration. Learning and updating the parameters at each iteration allows the method to converge on the true parameter values which may provide a more accurate result, but may be more computationally expensive.

In some embodiments, each of the one or more iterations further comprises, for each genetic variant determined to be causal, subtracting weighted effect sizes from the information about the association between each other genetic variant and the target phenotype of each input unit; the weighted effect sizes are the sampled effect size of the genetic variant on the target phenotype for the input unit weighted by respective correlation factors between the genetic variant and each other genetic variant; and the correlation factors are determined based on the information about correlations between the plurality of genetic variants in the region of interest. Subtracting the effect of a variant determined to be causal from linked variants ensures that multiple causal variants are not erroneously identified based on a single causal relationship. Using input-unit specific correlation factors allows the method to account for variations in genetic correlations between subpopulations.

In some embodiments, the input units are determined from respective groups of individuals, and the correlation factors between the genetic variant and each other genetic variant depend on an ancestry of the group of individuals of the input unit. In some embodiments, the group of individuals of at least one of the input units comprises individuals having a common ancestry, the correlation factors being determined based on correlations between genetic variants in the region of interest for individuals having the common ancestry. Using correlation factors based on ancestry is particularly useful, as individuals having different ancestries often have different patterns of correlation between genetic variants.

In some embodiments, the group of individuals of at least one of the input units comprises individuals having different ancestries, the correlation factors being determined based on an average of correlations between genetic variants in the region of interest for individuals having each of the different ancestries. Some input units may arise from studies that are not ancestry-stratified. Using a mixed set of correlation factors allows this data to still be incorporated in the method and improve results.

In some embodiments, the group of individuals of at least one of the input units comprises individuals having the same value of a characteristic. In some embodiments, the group of individuals of at least one of the input units comprises individuals having different values of a characteristic. In some embodiments, the characteristic is one of sex, age, weight, a molecular biomarker, or a behavioural characteristic. Subpopulations may also be defined based on characteristics, and input units based on data from individuals having those characteristics allow conclusions to be drawn about the differences in effect sizes between different subpopulations.

In some embodiments, carrying out one or more iterations comprises carrying out a predetermined number of iterations. Carrying out a predetermined number of iterations may provide adequate results for a known type of problem while remaining computationally efficient.

In some embodiments, each of the one or more iterations further comprises a step of evaluating a convergence parameter, and carrying out one or more iterations comprises carrying out iterations until a predetermined condition on the convergence parameter is met. Calculating a convergence parameter may be advantageous where an appropriate number of iterations is uncertain.

In some embodiments, the information about the association between the plurality of genetic variants and the target phenotype comprises, for each of the plurality of genetic variants, an estimate of a strength of association between the genetic variant and the target phenotype and an error in the estimate of the strength of association. As mentioned above, using this type of summary statistic data has advantages in the availability of large quantities of data.

According to another aspect, there is provided a method of determining a polygenic risk score for a target phenotype for a target individual comprising: receiving genetic information about a region of interest of the genome of the target individual; receiving prediction effect sizes on the target phenotype of a plurality of genetic variants in the region of interest determined using the method of analysing genetic data; and determining the polygenic risk score based on the genetic information for the target individual and the prediction effect sizes. As mentioned above, calculating polygenic risk scores is a particularly desirable use of the prediction effect sizes determined for genetic variants, and can be used for a variety of clinical applications. In some embodiments, the input units received in the method of analysing genetic data are determined from respective groups of individuals, and the polygenic risk score for the individual is determined using the prediction effect sizes for the input unit determined from a group of individuals most similar to the target individual. Using the prediction effect sizes for an input unit most appropriate to an individual can improve the accuracy of the polygenic risk score relative to one determined using generic effect sizes determined for unstratified data.

According to another aspect of the invention, there is provided an apparatus for analysing genetic data about an organism. The apparatus comprises a receiving unit configured to receive a plurality of input units, wherein each input unit comprises information about the association between a plurality of genetic variants in a region of interest of the genome of the organism and a target phenotype of the organism; and a data processing unit configured to: carry out one or more iterations comprising, for each of the plurality of genetic variants: determining whether the genetic variant is causal for the target phenotype based on the plurality of input units; and if the genetic variant is determined to be causal, determining a sampled effect size of the genetic variant on the target phenotype for each of the input units based on the plurality of input units and information about correlations between the plurality of genetic variants in the region of interest, the sampled effect size of the genetic variant on the target phenotype being non-zero for all of the input units, and for each genetic variant, determine a prediction effect size of the genetic variant on the target phenotype for each of the input units based on an average across at least a subset of the iterations of the sampled effect sizes of the genetic variant for the input unit or of posterior effect sizes of the genetic variant for the input unit calculated using the sampled effect sizes.

The invention may also be embodied in a computer program comprising instructions which cause the computer to carry out the method, or a computer-readable medium comprising instructions which, when executed by a computer, cause the computer to carry out the method.

Embodiments of the invention will be further described by way of example only with reference to the accompanying drawings, in which:

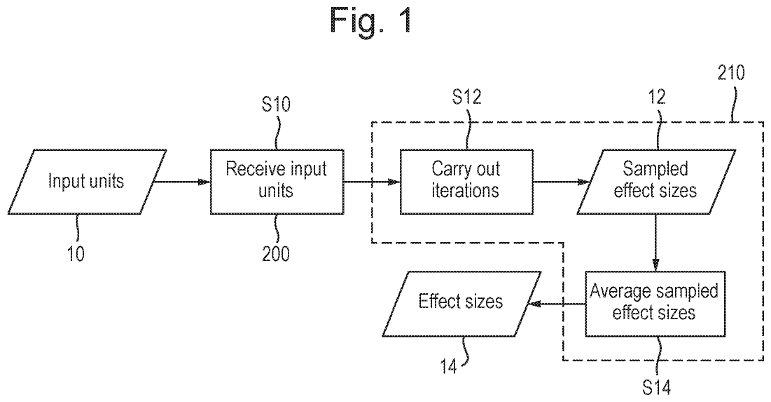

FIG. 1 is a flowchart of a method of analysing genetic data about an organism according to the invention;

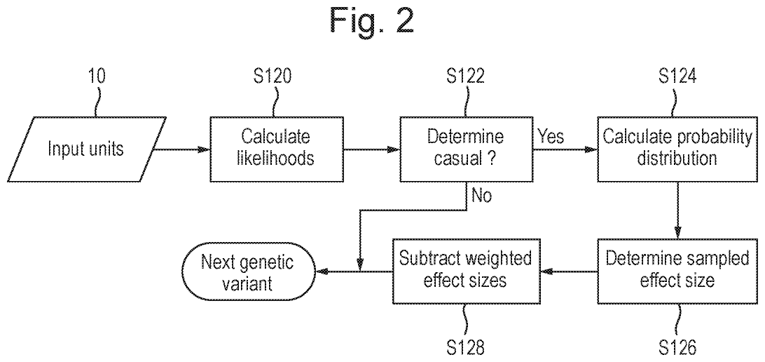

FIG. 2 is a flowchart showing the steps of each iteration in the step of carrying out iterations in the method of FIG. 1;

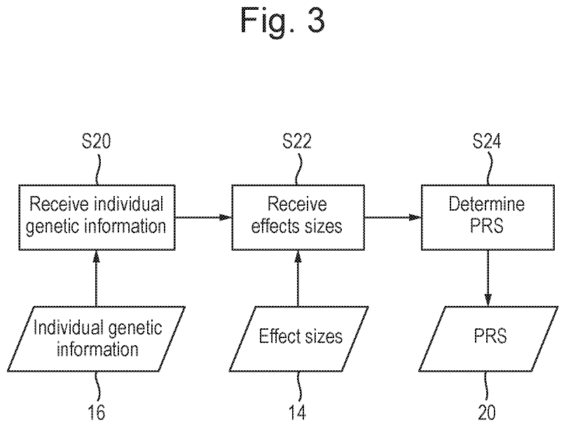

FIG. 3 is a flowchart of a method of determining a polygenic risk score according to the invention;

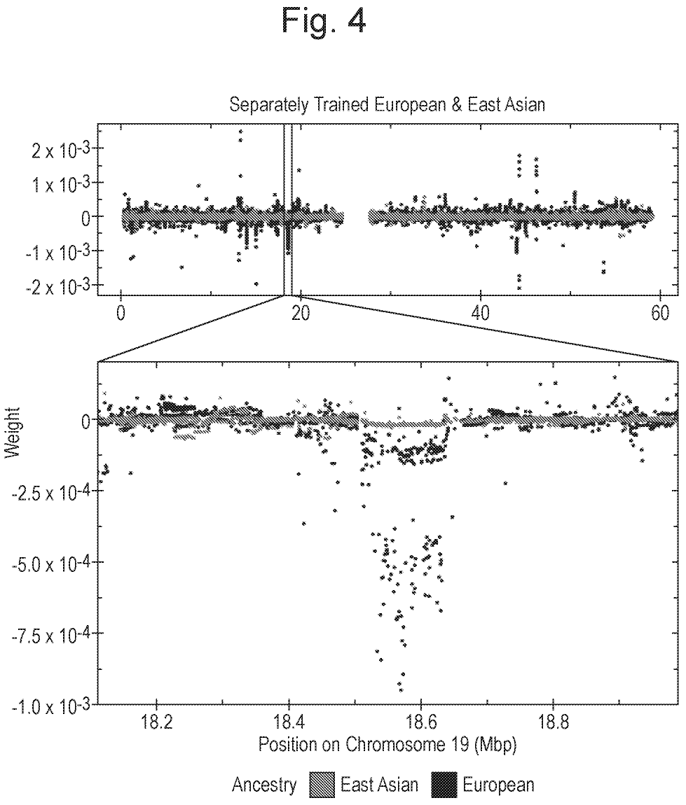

FIG. 4 is a graph showing effect sizes estimated for two different subpopulations using a prior art method of analysing genetic data; and

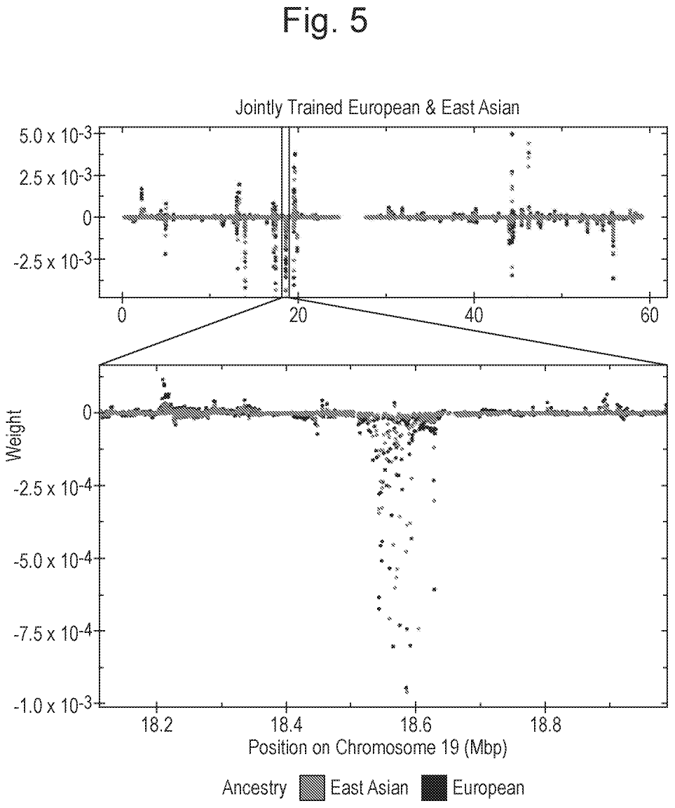

FIG. 5 is a graph showing effect sizes estimated for two different subpopulations using a method according to the invention.

FIG. 1 shows a computer-implemented method of analysing genetic data about an organism. Typically, the organism is a human, although the method may be applied to other organisms. Although the method refers to “an organism” this may not refer to a specific individual organism, but to the organism or a group of organisms generically.

The method comprises a step S10 of receiving a plurality of input units 10. The input units 10 comprise information about the association between a plurality of genetic variants in a region of interest of the genome of the organism and a target phenotype of the organism. The target phenotype may include any physical, behavioural, or other phenotypes that may be of interest. The genetic variants are typically single nucleotide polymorphisms, but may also comprise other types of genetic variation such as insertions or deletions of a section of the genome of the organism.

Each input unit 10 may be derived from one or more genome-wide association studies (GWAS), and so may also be referred to as a study or a GWAS. Each input unit 10 will comprise information about the association between the plurality of genetic variants and the target phenotype for a group of individuals, for example the individuals taking part in the corresponding GWAS.

At least a subset of the input units 10 are determined from a group of individuals from a particular subpopulation. For example, the group of individuals of at least one of the input units 10 may comprise individuals having a common ancestry. Alternatively or additionally, the group of individuals of at least one of the input units 10 may comprise individuals having the same value of a characteristic. The characteristic may be, for example, one of sex, age, weight, a molecular biomarker, or a behavioural characteristic such as whether the individuals smoke. In the case of continuous traits such as age or weight, the values of the characteristic may be divided into arbitrary bins to produce a discrete number of categories and divide the individuals for whom data is available into corresponding discrete groups with which to define the input units 10.

Since the definition of bins is not fixed by biology but is arbitrary, some embodiments of the method may comprise carrying out the steps of the method a plurality of times with different bin definitions (and correspondingly modified input units 10) and comparing the predictive power of the effect sizes generated with the different bin definitions. The effect sizes with the greatest predictive power may then be returned as the output of the method.

Not all of the input units 10 may be determined from a group of individuals from a particular subpopulation. For example, the group of individuals of at least one of the input units 10 may comprise individuals having different ancestries. Alternatively or additionally, the group of individuals of at least one of the input units 10 may comprise individuals having different values of a characteristic. Including one or more additional input units 10 from studies that are not subpopulation stratified can allow the method to leverage additional information from groups of individuals for which separation between subpopulations is not possible. This may be, for example, because the underlying data did not include information on particular characteristics of the individuals in the study, so it is not possible to stratify them.

In the embodiments described herein, the information about the association between the plurality of genetic variants and the target phenotype comprises, for each of the plurality of genetic variants, an estimate of a strength of association between the genetic variant and the target phenotype and an error in the estimate of the strength of association. Therefore, each input unit 10 comprises, for each variant i numbered 1 to n, an estimate \hat{β}ₗ of the strength of association between the variant i and the target phenotype, and a precision for that estimate, expressed as the standard error for the estimate SE ( [symbol] . This type of data is typically referred to as summary statistic data. However, in other embodiments, other types of information may be used, for example individual level data about all of the individuals in the groups from which the input units 10 are determined.

The estimates [symbol] of the strength of association in each input unit 10 are marginal effect sizes estimated from each variant independently in the GWAS study. A key challenge is a consequence of correlations between genetic variants in the population. The marginal effect sizes may include contributions that are in fact due to other, correlated genetic variants within the region of interest. For example, if variant a and variant b appear together very often, and variant b increases the risk of the target phenotype (i.e. is causal for the target phenotype), an effect may also be attributed to variant a, because it appears often in individuals with the target phenotype. Hence a single causal variant will generate significant associations at many other variants, themselves not causal but only correlated to the causal variant.

It is desirable to determine the unknown true effect size βᵢ (or strength of association) at each given variant i, which is adjusted for correlations with nearby variants. The problem of genetic prediction consists of estimating that set of true effect sizes βᵢ. While all the [symbol] values are typically different from 0, the number of non-zero βᵢ values will typically be much smaller. The challenge facing many methods of analysing genetic data therefore consists of identifying the subset of K truly causal variants Xᵢ and their true strength of association βᵢ. The number of causal variants K is in general unknown. That collection of causal variants and their corresponding true effect sizes (Xᵢ, βᵢ) can be used to calculate the polygenic risk score for the target phenotype.

In the present method, the estimation of which variants are causal and their corresponding effect sizes is achieved by exploring the space of possible (Xᵢ, βᵢ) in the step S12 of carrying out one or more iterations. The details of this step will be discussed further below. In some embodiments, carrying out one or more iterations comprises carrying out a predetermined number of iterations. This may be advantageous if it is known approximately how many iterations are needed to obtain an accurate result. In some embodiments, each of the one or more iterations further comprises a step of evaluating a convergence parameter, and carrying out one or more iterations comprises carrying out iterations until a predetermined condition on the convergence parameter is met. This may be advantageous if it is uncertain how many iterations will be required to give an accurate result.

As mentioned above, currently available methodologies for analysing genetic data (such as LDpred) consider one GWAS at a time and perform random sampling of which variants are causal, for example by Monte Carlo sampling. LDpred relies on being able to solve a Bayesian computation for one study and one genetic variant. It then uses a Gibbs sampling technique to extend the methodology from one to multiple correlated variants. Precisely, for a given genetic variant, LDpred uses a prior assumption that:
with probability (1-p) the effect of the genetic variant on the phenotype is 0 (i.e. the variant is not causal).
with probability p the effect on the outcome is normally distributed with mean 0 and variance a σ² (i.e. the variant is causal with a distribution of effect sizes centred around 0). With these assumptions, and the summary statistics [symbol] , SE ( [symbol] in a training GWAS for the relevant phenotype, it is possible to derive an analytical formula for the posterior distribution of the true effect size βᵢ on the target phenotype, and to sample from this distribution to estimate the true effect size.

However, this approach has limitations particularly for smaller studies that can lead to poor results for some subpopulations. For example, studies on individuals of non-European ancestry are less common and typically smaller than for those of European ancestry, leading to poor predictive results for individuals of non-European ancestry.

When considering multiple studies for the same target phenotype, currently available methods consist of combining the multiple studies into a single meta-analysis, and performing further processing, for example determining a PRS, on that meta-analysis. An example of a tool that accounts for evidence of association between variants and a target phenotype based on multiple studies is multi-trait analysis of GWAS (MTAG, Turley et al 2018). MTAG combines a set of GWAS and generates, for each input GWAS, a type of meta-analysis that results in updated summary statistics per input GWAS. These updated summary statistics can be fed into any standard PRS construction methodology, including LDPred (Craig et al, Nature Genetics 2020). However, MTAG uses the marginal effect sizes and standard errors without simultaneously accounting for LD information, meaning that the method is not fully leveraging the richness of the input datasets available. Another existing approach to combining multiple studies is the single variant Bayesian computation developed in another context (Trochet et al, Genetic Epidemiology 2019). In this method, the aim is not prediction of effect sizes, but the combining of studies to increase power to detect genetic associations. Hence, genetic variants are considered individually and there is no motivation to control for the correlation pattern between them.

The limitations of existing approaches can also be demonstrated by some example use cases.

In a first situation, a well powered GWAS exists in a first ancestry, typically individuals of European ancestries, for historical reasons. A second, less powered study, exists in another ancestry for the same target phenotype. The well-powered study cannot be readily combined with the second study using existing methods. Firstly, the correlation pattern between variants varies across ancestries, so the combination of two studies results in an undefined study that is challenging to analyse. Secondly, genetic and environmental differences across studies may result in either population specific variants or differences in effect sizes across these populations. Existing methods cannot account for this.

In a second situation, a predictive algorithm is to be generated that captures risk factors specific to subsets of a population. Current methods may fail to use the underlying genetic data to best advantage. It may be that “context-specific” PRSs calculated using effect sizes specific to a person's age, sex, ethnic group or any other social determinant of health) may be more accurate. For example, the determinants of cardiovascular disease (CVD) differ across genders, with differences in BMI, blood pressure, alcohol consumption and exercise patterns.

Existing methods solve this problem by acquiring samples that have already been stratified into subpopulation-specific studies, and then derive PRSs from these separately. For example, in the CVD example above, current methods would analyse GWAS for two gender specific cohorts (men and women) separately, and use each of these cohorts to generate a PRS. However, many of the genetic determinants are shared across genders. A joint analysis of the male and female cohorts, which would account for the sex differences and generate sex-specific PRS, would therefore be more appropriate to maximise predictive power. For example, if one is interested in a PRS for lung cancer in non-smokers, there is a similar choice with existing methods between 1) having many samples, that include smokers, or 2) using a smaller study that only consists of non-smokers.

However, the predictive ability of a PRS is also a function of the size of the underlying study. It is therefore generally detrimental to restrict the study samples to a subset of the data. In the smoking example, the first option uses a biased study (the PRS would suggest a larger effect size for addiction related variants from the proportion of the participants who are smokers), but the second option is likely to be underpowered (because 80% of lung cancer patients are smokers). This creates an opposing argument against subpopulation-specific PRSs.

These use cases are not exclusive of each other. One may, for example, wish to determine a PRS to predict a clinical outcome in a sex or socially-defined subset of a given ethnic group.

To overcome these limitations, the present method allows information from multiple studies to be combined when determining causal variants and their effect sizes, but, significantly, allows the determined effect sizes of each genetic variant to differ between input units 10. This allows the greater statistical power of larger studies to be used together with the data from smaller studies to improve estimations of which variants are causal in the smaller studies, but nonetheless different effect sizes for different subpopulations can be determined.

This involves extending the Bayesian computation from LDPred (Vilhjalmsson et al 2015) from one study to an arbitrary number of studies for the same phenotype, but in distinct subpopulations. In doing so, a link is made between the single variant multi-studies work of Trochet et al and the multi-variants single study work of Vilhjálmsson et al. By understanding the relationship between both methodological approaches, it becomes possible to integrate multiple studies in a flexible manner and to create a prediction algorithm based on multiple GWAS, rather than a single study.

As shown in FIG. 2, each iteration in the step S12 of the present method comprises, for each of the plurality of genetic variants, determining whether the genetic variant is causal for the target phenotype based on the plurality of input units 10. As for existing methods, genetic variants are considered one by one, for example in physical order or by random sampling, though other options are possible. However, at each variant, the present method incorporates multiple studies rather than a single study, and assesses the probability of models of the causality and effect size of the variant on each of the input units 10 (for example by Bayesian analysis, as discussed further below). Therefore, the present method determines whether each genetic variant is causal by analysing all of the input units 10 together, not by considering only one input unit 10 at a time, or by combining the input units 10 into a single meta-analysis as in existing methods.

If the genetic variant is determined to be causal, a step is performed of determining a sampled effect size 12 of the genetic variant on the target phenotype for each of the input units 10 based on the plurality of input units 10 and information about correlations between the plurality of genetic variants in the region of interest. Therefore, in the exploration of the space of causal variants and joint effect sizes, when a variant is selected as causal, different effect sizes are sampled for each study.

In the embodiment of FIG. 1, determining whether the genetic variant is causal comprises a step S120 of calculating a probability of the information from the plurality of input units assuming that the genetic variant is causal and a probability of the information from the plurality of input units assuming that the genetic variant is not causal, and a step S122 of stochastically determining the genetic variant to be causal with a probability dependent on a ratio of the probability of the information from the plurality of input units assuming that the genetic variant is causal and the probability of the information from the plurality of input units assuming that the genetic variant is not causal.

In step S120, the probability of the information from the plurality of input units assuming the genetic variant being causal may be dependent on a proportion of the plurality of genetic variants expected to be causal, the plurality of input units 10, and a correlation between the effect sizes of the genetic variant on the target phenotype for each of the input units 10. The probability of the information from the plurality of input units assuming that the genetic variant is not causal may be dependent on a proportion of the plurality of genetic variants expected to be causal, and the plurality of input units 10. The probabilities may be calculated using prior values.

For example, in an embodiment, two prior models are considered for any given variant:
a null hypothesis that, with probability (1-p), the variant has a 0 effect size for all input units 10; and
an alternative that, with probability p, the effect sizes of the genetic variant on the input unit 10 follow a multivariate Gaussian distribution.

The parameter p is the proportion of the plurality of genetic variants expected to be causal. In some embodiments, the proportion of the plurality of genetic variants expected to be causal is predetermined. This may be more computationally efficient if an estimate is available. In some embodiments, the proportion of the plurality of genetic variants expected to be causal is updated at each iteration. This allows the method to converge on the true value of p potentially improving the accuracy.

Under the null hypothesis, the value of the sampled effect size 12 is equal to 0 for all input units 10. Therefore, the covariance matrix for the sampled effect sizes βᵢ of the variant i is only driven by the uncertainty in the value of the parameters (referred to as SE_{i,j}, for the standard error of the marginal effect size of variant i from input unit j), which is itself a function of the sample size of the study and encoded in the summary statistics of the input unit 10. Precisely we have:

$${V}_{i} = [ \begin{matrix}{SE}_{i , 1}^{2} & 0 & 0 & 0 \\ 0 & {SE}_{i , 2}^{2} & 0 & 0 \\ 0 & 0 & … & 0 \\ 0 & 0 & 0 & {SE}_{i , m}^{2}\end{matrix} ] \qquad ( 1 )$$

where SE_{i,j} refers to the standard error for the variant i and input unit j, where there are m input units 10 in total.

Under the alternative, the sampled effect size βᵢ of the variant i is non-zero, and distributed as a multivariate Gaussian with mean 0 and a plurality of unknown variances σⱼ² for each dimension of the multivariate Gaussian. In the alternative, there is a new specification:

$${\tilde{\sum }}_{ι} = {\sum }_{i} + {V}_{i} \qquad ( 2 )$$

$${\sum }_{i} = [ \begin{matrix}{σ}_{1}^{2} & {ρ}_{i} {σ}_{1} {σ}_{2} & … & {ρ}_{i} {σ}_{1} {σ}_{m} \\ {ρ}_{i} {σ}_{1} {σ}_{2} & {σ}_{2}^{2} & … & {ρ}_{i} {σ}_{2} {σ}_{m} \\ … & … & … & … \\ {ρ}_{i} {σ}_{1} {σ}_{m} & {ρ}_{i} {σ}_{1} {σ}_{m} & … & {σ}_{m}^{2}\end{matrix} ] \qquad ( 3 )$$

with ρᵢ being the correlation between the effect sizes of the genetic variant i on the target phenotype for each of the m input units 10. In some embodiments, the correlation between the effect sizes of the genetic variant on the target phenotype for each of the input units 10 is predetermined. As for the proportion of variants expected to be causal, this may be more computationally efficient. The pre-determined value may be based on existing, external data if it allows an a priori estimation of how strongly the effects in different subpopulations should be correlated.

In other embodiments, the correlation between the effect sizes of the genetic variant on the target phenotype for each of the input units 10 is updated at each iteration. This allows the method to converge on the true parameter value, potentially leading to more accurate results. Alternatively, a grid of values of the correlation can be considered and the optimum parameter value for these correlations can be selected by maximising prediction in a dataset of individual level data with outcomes. In the example given here, the correlation between the effect sizes is a single parameter that is the same for all combinations of input units 10.

The correlation may also be a correlation matrix, allowing for the correlations to differ between different combinations of input units 10. For example, for continuous traits such as age, the correlation can be used for smoothing across bins of the variable. For a continuous trait like age, information can be borrowed from neighbouring age bins in order to improve the effect sizes and corresponding PRS for any given bin. Since there is an a priori expectation that neighbouring or nearby bins should have a higher genetic correlation than more distant bins, this could be accounted for using different values of the correlation between different bins of the continuous variable.

Once these two prior models are defined, the probability of the information from the plurality of input units assuming that the genetic variant is causal and the probability of the information from the plurality of input units assuming that the genetic variant is not causal can be calculated and combined with these priors.

In an embodiment of step S122, a Bayes factor can be calculated for each variant i using the probabilities determined in step S120:

$$Bayes factor = \frac{f ( {β}_{i} , {V}_{i} + {\sum }_{i} )}{f ( {β}_{i} , {V}_{i} )} \qquad ( 4 )$$

The stochastic sampling of whether the variant is causal is then performed based on the Bayes factor. βᵢ in these equations is a vector of dimension m, i.e. it specifies an effect of variant i on each of the m input units 10.

It is assumed that causal genetic variants are shared across input units 10 (and their corresponding subpopulations), and the effect size of these variants, while correlated across input units 10, varies. In other words, a variant is either causal for all of the input units 10 or for none of them. Therefore, if the genetic variant is determined to be causal, the sampled effect size 12 of the genetic variant on the target phenotype is determined to be non-zero for all of the input units 10.

Where the input units 10 are determined from respective groups of individuals, and depending on the studies that are used to determine the input units 10, one potential issue is sample overlap across studies. For example, a “combined genders” study may be used to derive one input unit 10, and is consequently analysed jointly with input units 10 derived from other “male only” and “female only” studies. The male and female specific studies may be subsets of the larger combined gender study set, while the combined gender study may include additional samples, for which gender information was not provided, or simply be the union of the two gender-specific studies. To account for this, in some embodiments, the probability of the information from the plurality of input units assuming that the genetic variant being causal is dependent on one or more parameters quantifying an overlap in the groups of individuals between respective pairs of input units 10.

For example, one way to account for that possibility is to update the covariance matrix Vᵢ shown above to become:

$${V}_{i} = [ \begin{matrix}{SE}_{i , 1}^{2} & {r}_{1 , 2} {SE}_{i , 1} {SE}_{i , 2} & … & {r}_{1 , m} {SE}_{i , 1} {SE}_{i , m} \\ {r}_{1 , 2} {SE}_{i , 1} {SE}_{i , 2} & {SE}_{i , 2}^{2} & … & {r}_{2 , m} {SE}_{i , 1} {SE}_{i , m} \\ … & … & … & … \\ {r}_{1 , m} {SE}_{i , 1} {SE}_{i , m} & {r}_{2 , m} {SE}_{i , 1} {SE}_{i , m} & … & {SE}_{i , m}^{2}\end{matrix} ] \qquad ( 5 )$$

where the r_{x,y} coefficients account for the overlap in samples across studies, and (as will be discussed further below) also model correlations across the sampled effect sizes 12 due to sharing of samples. To clarify notations, these r_{x,y} have no relationship to the correlation factors r_{i,j} describing variant level correlation (which will be discussed in more detail below). This addition (described in Trochet et al 2019) is important in practice to achieve accurate results, although it is not essential and adequate results may still be achieved without it.

If the genetic variant is determined to be causal, a posterior mean and variance can be computed for the joint effect size across all input units 10. The step of determining a sampled effect size 12 of the genetic variant comprises a step S124 of calculating a probability distribution of effect sizes of the genetic variant on the target phenotype for the input units 10, and a step S126 of sampling values of the effect sizes for the input units 10 from the probability distribution.

A sampled effect size 12 is used because in practice it is impossible to fully explore the space of all possible causal variants and all possible corresponding effect sizes in a reasonable time. Therefore, sampling techniques, for example Monte Carlo simulations, are used to explore the space of causal variants and their corresponding effect sizes. In some embodiments, the sampling of values of the effect size in each iteration is dependent on the sampled effect sizes 12 from one or more previous iterations. This can be used to guide the sampling technique to adequately explore the space of possible values. In some embodiments, the sampling of values of the effect size is performed using a Monte-Carlo Gibbs sampler.

In a preferred embodiment, the probability distribution is a multivariate normal distribution. The probability distribution may be dependent on a correlation between the effect sizes of the genetic variant on the target phenotype for each of the input units 10. As discussed for the probabilities above, the correlation between the effect sizes of the genetic variant on the target phenotype for each of the input units 10 may be predetermined. Alternatively, the correlation between the effect sizes of the genetic variant on the target phenotype for each of the input units 10 may be updated at each iteration, allowing the method to learn a suitable value of the correlation.

In a specific example, the probability distribution is the posterior mean for the effect size, and is distributed as a multivariate normal distribution:

βᵢ˜MVN((Σᵢ⁻¹+Vᵢ⁻¹)⁻¹(Vᵢ⁻¹βᵢ), (Σᵢ⁻¹+Vᵢ⁻¹)⁻¹) (6)

An important step in some embodiments of methods for analysing genetic data with the aim of calculating a PRS is the ability to control for correlations between genetic variants. As mentioned above, correlations between variants can cause some variants to have large marginal effect sizes even when they are not causal for the target phenotype.

To account for this, in some embodiments, each of the one or more iterations further comprises, for each genetic variant determined to be causal, a step S128 of subtracting weighted effect sizes from the information about the association between each other genetic variant and the target phenotype of each input unit 10. Hence when a genetic variant i is determined to be causal, and a sampled effect size βᵢ is determined for the genetic variant i, the effect of that causal variant is subtracted from surrounding correlated variants. The weighted effect sizes are the sampled effect size 12 of the genetic variant on the target phenotype for the input unit 10 weighted by respective correlation factors between the genetic variant and each other genetic variant.

In a particular embodiment, this results in the following correction being applied to the marginal effect sizes of each of the other genetic variants j:

$${β}_{j}^{corrected} = {\hat{β}}_{j} - {\sum }_{i} {r}_{i , j} {β}_{i} \qquad ( 7 )$$

In the formula above, the βᵢ are the sampled effect sizes 12 of each of the variants currently determined to be causal. The values r_{i,j} are correlation factors that describe the correlation between each pair of variants i and j. The correlation factors are determined based on the information about correlations between the plurality of genetic variants in the region of interest, which may be estimated from a reference set of reference sequences. This correction formula assumes that each genotyped variant Xᵢ has been normalized to have variance 1, and that its associated marginal effect size \hat{β}ₗ has been updated accordingly. If this is not the case, an additional correction needs to be applied to account for the standard error for each estimated effect size.

The effect of this correction is that, when it is determined whether a variant is causal, its marginal effect size will be corrected using the formula above based on the sampled effect sizes of all the variants so far determined to be causal in that iteration. Therefore, in such embodiments, the effect size βᵢ used in equations (4) and (6) will actually be the corrected effect size calculated using equation (7). A significant subtlety is that this subtraction step for a particular genetic variant depends on which other variants have been sampled as causal at the point the subtraction is performed. Therefore, some variation in βᵢ can arise between iterations depending on the order in which genetic variants are sampled.

Importantly, it is often not possible to calculate the correlation factors between genetic variants (the values r_{i,j} in the example above) directly from the data itself and instead must originate from a reference population, such as data generated by the 1,000 Genomes consortium. The set of these correlation factors may be referred to as a linkage disequilibrium map (or LD map), and reflects a covariance structure between the genetic variants. As mentioned above, these correlation factors may vary between subpopulations, for example for different ancestries. In existing methods, which only analyse a single study those correlation factors will be determined from a reference population LD map matching the population of origin for the study.

However, in the present method, a challenge is dealing with the effect size subtraction step S128 that accounts for correlations across genetic variants in a manner that is consistent with ancestry-specific patterns of variant correlations. To overcome this challenge, the present method may, where appropriate, handle in parallel multiple reference LD maps. Once a variant is determined to be causal, the subtraction step S128 is then applied in an ancestry specific manner. Therefore, where the input units 10 are determined from respective groups of individuals, the correlation factors between the genetic variant and each other genetic variant depend on an ancestry of the group of individuals of the input unit 10. A one-to-one mapping may be used between the ancestry where each study was performed and its matching LD map (covariance structure).

For example, where the group of individuals of at least one of the input units 10 comprises individuals having a common ancestry, the correlation factors are determined based on correlations between genetic variants in the region of interest for individuals having the common ancestry.

In another example, the plurality of input units 10 are derived from studies that contain individuals from a mixture of ancestries. Where the group of individuals of at least one of the input units 10 comprises individuals having different ancestries, the correlation factors are determined based on an average of correlations between genetic variants in the region of interest for individuals having each of the different ancestries. The method determines the LD map for the mixed input units 10 to be an average of plural “primary” LD maps, each of these “primary” LD maps being determined from a well-defined reference ancestry set of correlations between genetic variants.

Where the group of individuals of the input units 10 have a common ancestry, but have different values of another characteristic such as gender, it may not be necessary to handle multiple LD maps simultaneously, as the single LD map for the common ancestry is sufficient.

It is possible that, depending on the input data used, not all of the plurality of genetic variants may exist at meaningful frequencies for all ancestries. For example, some genetic variants may only be found in individuals of a specific ancestry. When this is the case, and a causal effect is assigned to one of these low-frequency variants, it may be assumed that this variant absent in a given ancestry is uncorrelated with other variants for the same ancestry. Therefore, the r_{i,j} correlation factors for the correlation between the low-frequency variants and all other variants may be set to zero.

Once the one or more iterations have been completed, the method comprises a step S14 of, for each genetic variant, determining a prediction effect size 14 of the genetic variant on the target phenotype for each of the input units 10 based on an average of the sampled effect sizes 12 of the genetic variant for the input unit 10. The prediction effect size 14 may also be based on an average of posterior effect sizes of the genetic variant for the input unit calculated using the sampled effect sizes 12. The average in either case is taken across at least a subset of the iterations. Any suitable method for averaging may be used. Using multiple iterations and averaging the results overcomes the randomness of the effect size sampling. Once the set of causal variants and their effect sizes 14 has been determined, it becomes straightforward to determine a PRS based on the effect sizes 14. In an embodiment, the average of the sampled effect sizes may be a weighted average, where the sampled effect size of each variant determined to be causal is weighted by a posterior probability that the variant is causal.

For example, the average effect size βᵢ for variant i may be calculated as:

$$\underline{{β}_{i}} = \frac{1}{L} {\sum }_{l = 1}^{L} {p}_{i , l} {β}_{i , l} \qquad ( 8 )$$

where L denotes the total number of iterations, optionally after some initial burn in iterations. The posterior probability that the variant is causal can be determined in any suitable way. For example, it may be determined using the number of iterations in which the variant was determined to be causal, as a proportion of the total number of iterations carried out. Alternatively, the posterior probability that the variant is causal may be calculated from the probability of the information from the plurality of input units assuming that the variant is causal when calculating the Bayes factor using the ratio of probabilities as shown, for example, in equation (4)ƒ (βᵢ, Vᵢ+Σᵢ).

Keeping with the example of smoking in lung cancer, the present method allows an input unit 10 derived from a large lung cancer GWAS (not stratified by smoking status) to be analysed jointly with an input unit 10 derived from a smaller lung cancer GWAS in non-smokers. This will effectively result in two sets of prediction effect sizes 14 for the phenotype of lung cancer in two subpopulations, namely non-smokers and the general population. For most genetic variants, the prediction effect sizes 14 would be the same for both input units 10 corresponding to the two subpopulations. However, for addiction-related variants, the effect sizes for the input unit 10 from the smaller GWAS should clearly show that these variants are not related to lung cancer in non-smokers. This effectively achieves the above-mentioned goal of allowing a lung cancer PRS to be obtained for which addiction-related variants have been subtracted.

Typically, the method performs best if the variation in the size of the groups of individuals from which the input units 10 are determined is not too large. For example, when two input units 10 are used derived from a smaller and a larger group of individuals, a significant performance improvement is generally observed once that the smaller group of individuals is about ˜20% or greater of the size of the larger group of individuals.

In some embodiments, one or more of the sampled effect sizes 12 for each genetic variant may be discarded and not included in the average used to obtain the prediction effect sizes 14. The number not included may be predetermined, or based on the value of the sampled effect size 12. The discarded sampled effect sizes 12 may be those from the first iterations of the method, for example the first ten iterations, the first twenty iterations, or some other predetermined number. These are often referred to as “burn-in” iterations, and are usually discarded because sampling techniques such as a Monte-Carlo Gibbs sampler take several iterations to converge to a useful sampling pattern.

Given the desirability of determining PRS in general, the present invention can also be used in a method of determining a polygenic risk score for a target phenotype for a target individual, as illustrated in FIG. 3. The improved estimates of prediction effect sizes obtained using the methods described above allow for the determination of more accurate PRSs.

The method of determining a PRS comprises a step S20 of receiving genetic information 16 about a region of interest of the genome of the target individual. This may comprise information about the genetic variants (such as single-nucleotide polymorphisms, deletions of insertions) expressed by the individual in the region of interest.

The method further comprises a step S22 of receiving prediction effect sizes 14 on the target phenotype of a plurality of genetic variants in the region of interest determined using the method of analysing genetic data described above.

The method further comprises a step S24 of determining the polygenic risk score based on the genetic information for the target individual 16 and the effect sizes 14.

In an embodiment, the input units 10 received in the method of analysing genetic data are determined from respective groups of individuals, and the polygenic risk score 20 for the individual is determined using the prediction effect sizes 14 for the input unit 10 determined from a group of individuals most similar to the target individual. For example, if the effect sizes 14 are determined for two input units 10 determined from groups of individuals having European and East-Asian ancestry respectively, and the individual is of East-Asian ancestry, the prediction effect sizes 14 for the East-Asian input unit 10 would be used to determine the PRS 20 for the individual.

In an embodiment, the PRS 20 is calculated as follows:

$$PRS = {\sum }_{k = 1}^{K} {α}_{k} {x}_{k} \qquad ( 9 )$$

where K is the number of variants that contribute to the PRS 20, xₖ is the genotype for variant k, and αₖ is the PRS weight for variant k, which quantifies the predictive impact of variant k on the target phenotype (i.e. quantifying the strength of association of variant l on the target phenotype). Typically the PRS weight αₖ is simply the average effect size for variant k as calculated above, i.e. βₖ.

The method of analysing genetic data may be carried out by an apparatus for analysing genetic data about an organism, also illustrated in FIG. 1. The apparatus comprises a receiving unit 200 configured to receive a plurality of input units 10, wherein each input unit comprises information about the association between a plurality of genetic variants in a region of interest of the genome of the organism and a target phenotype of the organism. The apparatus further comprises a data processing unit 210 configured to carry out one or more iterations comprising, for each of the plurality of genetic variants, determining whether the genetic variant is causal for the target phenotype based on the plurality of input units, and if the genetic variant is determined to be causal, determining a sampled effect size 12 of the genetic variant on the target phenotype for each of the input units 10 based on the plurality of input units 10 and information about correlations between the plurality of genetic variants in the region of interest. The sampled effect size 12 of the genetic variant on the target phenotype is non-zero for all of the input units 10. The data processing unit 210 is further configured to, for each genetic variant, determine a prediction effect size 14 of the genetic variant on the target phenotype for each of the input units 10 based on an average across at least a subset of the iterations of the sampled effect sizes 12 of the genetic variant for the input unit 10 or of posterior effect sizes of the genetic variant for the input unit 10 calculated using the sampled effect sizes 12.

The invention may also be embodied in a computer program comprising instructions which, when the program is executed by a computer, cause the computer to carry out the method of analysing genetic data. The invention may also be embodied in a computer-readable medium comprising instructions which, when executed by a computer, cause the computer to carry out the method of analysing genetic data.

### Results

### Cross-Ancestry

To illustrate the effectiveness of the present method in determining effect sizes for subpopulations of different ancestries, examples of effect sizes determined using a prior art method are shown in FIG. 4, and effect sizes determined using the present method are shown in FIG. 5.

Well-powered breast cancer summary statistics data exist for individuals of European ancestry, and much smaller cohorts exist for East-Asian women, as evidenced by the differential in the number of cases (Table 1). In addition, two well powered cohorts are available to evaluate effect size for various phenotypes: the UK Biobank (Bycroft et al) for European ancestries individuals and the multi ethnic cohort (MEC) for individuals of East Asian ancestries.

FIGS. 4 and 5 both shown inferred effect sizes of genetic variants on chromosome 19 for two input units determined from two breast cancer studies on individuals of East Asian ancestry (red) and individuals of European ancestry (black). FIG. 4 shows the effect sizes when determined separately for the two input units using a prior art method. FIG. 5 shows the effect sizes when determined jointly for the two input units using the present method.

When effect sizes are determined by analysing each input unit separately (FIG. 4), genetic variants at the established cancer locus ELL (zoomed insert in lower panel of FIGS. 4 and 5) have large weights for Europeans. However, the smaller sample size in the study on individuals of East Asian ancestry is not sufficient to detect this signal. When effect sizes are determined by analysing the input units jointly (FIG. 5), the combination of both studies provides enough statistical power for East Asians to also have large effect sizes at the established cancer locus ELL.

Genome-wide, joint analysis using the present method improves prediction performance for both ancestries. In addition, the joint analysis significantly changes the accuracy with which causal variants are located. This can be observed in FIGS. 4 and 5, where the large non-zero effect sizes for breast cancer in both European and East-Asian ancestries span much shorter locational distances in the top panel of FIG. 5 (joint analysis) than in the top panel of FIG. 4 (separate analysis). This reflects a better understanding of the localisation of causal variants obtained by combining data from multiple ancestries.

Table 1 shows the training populations used to determine a breast cancer PRS in women of European and East Asian ancestries.

TABLE 1

| Ethnicity | Number of cases | Number of controls |
|---|---|---|
| European ancestries | 122,977 | 105,974 |
| East Asia | 21,098 | 108,814 |
| Japan | 5,552 | 89,731 |
| Korea | 1,478 | 5,979 |

With these cohorts, the PRS predictive ability was evaluated for different methods of determining the prediction effect sizes used in PRS calculation, namely LDPred, MTAG, and the present method. The results are shown in Table 2, where bold font indicates the best performance for each ancestry. Because breast cancer is a binary trait, the area under the curve (AUC) is used as a measure of predictive accuracy to quantify the separation of the PRS between breast cancer cases and controls. The best performing method was the present method, which combines input units from studies from multiple ancestries and generates ancestry-specific versions of the PRS based on the effect sizes for each input unit.

TABLE 2

| | Performance (AUC) | Performance (AUC) |
|---|---|---|
| Methodology | East-Asian | European |
| LDPred, European PRS | 0.614 | 0.654 |
| LDPred, East-Asian PRS | 0.615 | 0.579 |
| MTAG, European PRS | 0.612 | 0.654 |
| MTAG, East-Asian PRS | 0.616 | 0.579 |
| Present method, | 0.626 | 0.660 |
| European PRS | | |
| Present method, | 0.646 | 0.640 |
| East-Asian PRS | | |

### Context-Specific

As discussed above, the present method can also be used to determine prediction effect sizes specific to subpopulations determined based on other characteristics of individuals. Different strata of the population can be handled in a manner similar to different ancestries, and PRS specific to these different strata can also be calculated. In the example below, it is assumed that the studies used to determine the input units originate from a single population. Therefore, there is no need to consider different sets of correlation factors (i.e. LD maps describing the correlation structure between genetic variants) for each input unit. However, as mentioned above, there is a chance that there may be overlap in the samples of individuals between studies.

In this example, the prediction effect sizes of genetic variants on BMI are determined on input units determined using a training data set from GIANT consortium GWAS, (152,893 males, 171,977 females, or 332,154 combined). The PRS obtained from the effect sizes are then applied to an evaluation data set. Because BMI is a quantitative trait, the percentage of variance explained (r²) is used as a measure of predictive accuracy. A comparison is performed between PRS from effect sizes generated using two approaches:
an approach using an existing method that combines both genders into a single meta-analysis and generates a single PRS that is evaluated in both men and women; and
the present method that jointly analyses a BMI study in men and another BMI study in women, and generates different effect sizes and two distinct PRS (one per gender).

The results of this comparison are shown in Table 3. Bold font indicates the best performing methodology for each of the two genders.

TABLE 3

| | Evaluation Set | | |
|---|---|---|---|
| Source of effect sizes | Male | Female | Combined |
| Present method - male effect sizes | 9.71 | 8.26 | 8.72 |
| from sex-stratified analysis | | | |
| Present method - female effect sizes | 9.58 | 8.75 | 8.96 |
| from the sex stratified analysis | | | |
| Existing method - combined gender meta- | 9.25 | 8.15 | 8.48 |
| analysis, without sex stratification | | | |

The BMI variance explained is higher for males when using the male effect sizes from the present, sex stratified approach. Similarly, the BMI variance explained is higher for females when using the female weights effect sizes from the present, sex stratified approach. In both cases, a meta-analysis of male and female using existing methods does not perform as well. In addition, using either of the male and female effect sizes from the present method explains a higher percentage of the BMI variance in the combined gender evaluation set than the existing meta-analysis-based method.

### REFERENCES

Bayesian meta-analysis across genome-wide association studies of diverse phenotypes, Trochet H, Pirinen M, Band G, Jostins L, McVean G, Spencer C, Genetic Epidemiology 2019

Multi-trait analysis of genome-wide association summary statistics using MTAG, P Turley et al. Nature Genetics 2018

Vilhjálmsson B J, Yang J, Finucane H K, et al. Modeling Linkage Disequilibrium Increases Accuracy of Polygenic Risk Scores. Am J Hum Genet 2015.

Variable prediction accuracy of polygenic scores within an ancestry group, Hakhamanesh Mostafavi, Arbel Harpak Ipsita Agarwal, Dalton Conley, Jonathan K Pritchard, Molly Przeworski, eLife, 2020

Bycroft et al, The UK Biobank resource with deep phenotyping and genomic data, Nature 2018

A correction for sample overlap in genome-wide association studies in a polygenic pleiotropy-informed framework, Marissa LeBlanc, Verena Zuber, Wesley K. Thompson, Ole A. Andreassen, Schizophrenia and Bipolar Disorder Working Groups of the Psychiatric Genomics Consortium, Arnoldo Frigessi, and Bettina Kulle Andreassen, 2018

Multitrait analysis of glaucoma identifies new risk loci and enables polygenic prediction of disease susceptibility and progression, Jamie E. Craig et al, Nature Genetics 2020

1. A computer-implemented method of analysing genetic data about an organism, the method comprising:
receiving a plurality of input units, wherein each input unit comprises information about the association between a plurality of genetic variants in a region of interest of the genome of the organism and a target phenotype of the organism;
carrying out one or more iterations comprising, for each of the plurality of genetic variants:
determining whether the genetic variant is causal for the target phenotype based on the plurality of input units; and
if the genetic variant is determined to be causal, determining a sampled effect size of the genetic variant on the target phenotype for each of the input units based on the plurality of input units and information about correlations between the plurality of genetic variants in the region of interest, the sampled effect size of the genetic variant on the target phenotype being non-zero for all of the input units; and
for each genetic variant, determining a prediction effect size of the genetic variant on the target phenotype for each of the input units based on an average across at least a subset of the iterations of the sampled effect sizes of the genetic variant for the input unit or of posterior effect sizes of the genetic variant for the input unit calculated using the sampled effect sizes.

2. The method of claim 1, wherein determining whether the genetic variant is causal comprises calculating the probability of the information from the plurality of input units, assuming that the genetic variant is causal and a probability of the information from the plurality of input units assuming that the genetic variant is not causal, and stochastically determining the genetic variant to be causal with a probability dependent on a ratio of the probability of the input data assuming that the genetic variant is causal and the probability of the input data assuming that the genetic variant is not casual.

3. The method of claim 2, wherein the probability of the information from the plurality of input units assuming that the genetic variant is causal is dependent on:
a proportion of the plurality of genetic variants expected to be causal;
the plurality of input units; and
a correlation between the effect sizes of the genetic variant on the target phenotype for each of the input units.

4. The method of claim 2, wherein the probability of the information from the plurality of input units assuming that the genetic variant is not causal is dependent on:
a proportion of the plurality of genetic variants expected to be causal; and
the plurality of input units.

5. The method of claim 3, wherein the proportion of the plurality of genetic variants expected to be causal is predetermined.

6. The method of claim 3, wherein the correlation between the effect sizes of the genetic variant on the target phenotype for each of the input units is predetermined.

7. The method of claim 3, wherein the proportion of the plurality of genetic variants expected to be causal is updated at each iteration.

8. The method of claim 3, wherein the correlation between the effect sizes of the genetic variant on the target phenotype for each of the input units is updated at each iteration.

9. The method of claim 2, wherein the input units are determined from respective groups of individuals, and the probability of the information from the plurality of input units assuming that the genetic variant being causal is dependent on one or more parameters quantifying an overlap in the groups of individuals between respective pairs of input units.

10. The method of claim 1, wherein determining the sampled effect size of the genetic variant comprises calculating a probability distribution, for example a multivariate normal distribution, of effect sizes of the genetic variant on the target phenotype for the input units, and sampling values of the effect sizes for the input units from the probability distribution.

11. (canceled)

12. The method of claim 10, wherein the sampling of values of the effect size in each iteration is dependent on the sampled effect sizes from one or more previous iterations.

13. The method of claim 10, wherein the sampling of values of the effect size is performed using a Monte-Carlo Gibbs sampler.

14. The method of claim 10, wherein the probability distribution is dependent on a correlation between the effect sizes of the genetic variant on the target phenotype for each of the input units. (Currently Amended) The method of claim 14, wherein the correlation between the effect sizes of the genetic variant on the target phenotype for each of the input units is either predetermined or updated at each iteration.

16. (canceled)

17. The method of claim 1, wherein:
each of the one or more iterations further comprises, for each genetic variant determined to be causal, subtracting weighted effect sizes from the information about the association between each other genetic variant and the target phenotype of each input unit;
the weighted effect sizes are the sampled effect size of the genetic variant on the target phenotype for the input unit weighted by respective correlation factors between the genetic variant and each other genetic variant; and
the correlation factors are determined based on the information about correlations between the plurality of genetic variants in the region of interest.

18. The method of claim 17, wherein the input units are determined from respective groups of individuals, and the correlation factors between the genetic variant and each other genetic variant depend on an ancestry of the group of individuals of the input unit.

19. The method of claim 18, wherein the group of individuals of at least one of the input units comprises individuals having a common ancestry, the correlation factors being determined based on correlations between genetic variants in the region of interest for individuals having the common ancestry.

20. The method of claim 18, wherein the group of individuals of at least one of the input units comprises individuals having different ancestries, the correlation factors being determined based on an average of correlations between genetic variants in the region of interest for individuals having each of the different ancestries.

21. The method of claim 1, wherein the group of individuals of at least one of the input units comprises one or both of individuals having the same value of a characteristic and individuals having different values of a characteristic.

22. (canceled)

23. The method of claim 21, wherein the characteristic is one of sex, age, weight, a molecular biomarker, or a behavioural characteristic.

24. The method of claim 1, wherein carrying out one or more iterations comprises carrying out a predetermined number of iterations.

25. The method of claim 1, wherein each of the one or more iterations further comprises a step of evaluating a convergence parameter, and carrying out one or more iterations comprises carrying out iterations until a predetermined condition on the convergence parameter is met.

26. The method of claim 1, wherein the information about the association between the plurality of genetic variants and the target phenotype comprises, for each of the plurality of genetic variants, an estimate of a strength of association between the genetic variant and the target phenotype and an error in the estimate of the strength of association.

27. A method of determining a polygenic risk score for a target phenotype for a target individual comprising:
receiving genetic information about a region of interest of the genome of the target individual;
receiving prediction effect sizes on the target phenotype of a plurality of genetic variants in the region of interest determined using the method of analysing genetic data of claim 1; and
determining the polygenic risk score based on the genetic information for the target individual and the prediction effect sizes.

28. The method of claim 27, wherein the input units received in the method of analysing genetic data are determined from respective groups of individuals, and the polygenic risk score for the individual is determined using the prediction effect sizes for the input unit determined from a group of individuals most similar to the target individual.

29. An apparatus for analysing genetic data about an organism, the apparatus comprising:
a receiving unit configured to receive a plurality of input units, wherein each input unit comprises information about the association between a plurality of genetic variants in a region of interest of the genome of the organism and a target phenotype of the organism; and
a data processing unit configured to:
carry out one or more iterations comprising, for each of the plurality of genetic variants:
determining whether the genetic variant is causal for the target phenotype based on the plurality of input units; and
if the genetic variant is determined to be causal, determining a sampled effect size of the genetic variant on the target phenotype for each of the input units based on the plurality of input units and information about correlations between the plurality of genetic variants in the region of interest, the sampled effect size of the genetic variant on the target phenotype being non-zero for all of the input units; and
for each genetic variant, determine a prediction effect size of the genetic variant on the target phenotype for each of the input units based on an average across at least a subset of the iterations of the sampled effect sizes of the genetic variant for the input unit or of posterior effect sizes of the genetic variant for the input unit calculated using the sampled effect sizes.

30. A computer program or a computer-readable medium comprising instructions which, when executed by a computer, cause the computer to carry out the method of claim 1.

31. (canceled)
